(19) United States
(12) Patent Application Publication
(10) Pub. No.: US 2013/0005824 A1
(43) Pub. Date: Jan. 3, 2013

(54) TREATMENT OF ISCHEMIC TISSUE

(75) Inventors: Richard A. Clark, Poquott, NY (US); Adam Singer, Setauket, NY (US); Mary Frame McMahon, Sag Harbor, NY (US)

(73) Assignee: The Research Foundation of State University of New York, Albany, NY (US)

(21) Appl. No.: 13/502,196

(22) PCT Filed: Oct. 15, 2010
(86) PCT No.: PCT/US10/52906

§ 371 Date: Aug. 6, 2012

Related U.S. Application Data
(60) Provisional application No. 61/252,415, filed on Oct. 16, 2009.

(51) Int. Cl. A61K 31/12; A61P 17/02; A61P 9/10

(52) U.S. Cl. 514/679

(57) ABSTRACT

The present invention features methods of treating patient for ischemic tissue damage. The methods can be carried out by administering (e.g., intravenously administering) a curcuminoid or a pharmaceutically active salt, metabolite, or analog thereof at low doses.

### CROSS-REFERENCE TO RELATED APPLICATIONS

This application claims the benefit of the filing date of U.S. Provisional Application No. 61/252,415, which was filed on Oct. 16, 2009. For the purpose of any U.S. application that may claim the benefit of U.S. Provisional Application No. 61/252,415, the content of that earlier-filed provisional application is hereby incorporated by reference in its entirety.

### FEDERALLY SPONSORED RESEARCH OR DEVELOPMENT

This invention was made with government support awarded by The Armed Forces Institute of Regenerative Medicine (AFIRM) under grant numbers S1031970 and W81XWH-08-2-0034. The government has certain rights in the invention.

### TECHNICAL FIELD

This invention relates to compositions and methods for the treatment of ischemic tissue damage, and more particularly to formulations containing curcuminoids that can be used at low doses to limit ischemic damage in tissues such as the skin, myocardium, and central nervous system.

### BACKGROUND

Curcumin is a chemical substance within the spice turmeric (from Curcuma longa), which has been used for centuries to treat a wide variety of inflammatory conditions. Studies have shown that curcumin possesses many biological activities including anti-inflammatory, anti-oxidant, and anti-microbial action (see Maheshwari et al., Life Sciences 78:2081-2087, 2006).

Curcumin is a potent scavenger of free oxygen radicals including superoxide anion radicals, hydroxyl radicals and nitrogen dioxide radicals. It also inhibits lipid peroxidation and oxidative cell injury. Curcumin has been shown to reduce the proliferation and contraction of keloid and hypertrophic scar-derived fibroblasts in vitro. Either oral or topically administered curcumin enhanced healing in full-thickness skin wounds in both normal rats, guinea pigs and pigs as well as in streptozotocin-induced diabetic rats. Treatment of wounds with curcumin enhanced expression of fibronectin and collagen and increased the formation of granulation tissue while promoting neovascularization and faster re-epithelialization.

### SUMMARY

The present invention is based, in part, on our studies of burn injury progression. We asked whether intravenous therapy with curcumin I (which is also known as diferuloylonethane) could impede burn injury progression, and we also studied changes in vascular diameter following local application of curcumin in hamster cheek pouch tissue. We discovered that curcumin, when administered in low doses, could cause vasodilation and was efficacious in treating burn injury.

Accordingly, the invention features methods of treating patients for ischemic tissue damage. The methods can be carried out by administering (e.g., intravenously administering) a curcuminoid or a pharmaceutically active salt, metabolite, or analog thereof at low doses (including doses that are, to our knowledge, much lower than those previously suggested). Below, we characterize the dosage in at least two ways. The first is by the amount of the curcuminoid, by weight, that is delivered to the patient. Absolute amounts can vary depending on one or more characteristics concerning the patient (e.g., the patient's weight, metabolic state, age, and gender). The second is by the concentration of the curcuminoid in the circulation and/or at the site of ischemic tissue damage after administration of the curcuminoid. The concentration can be assessed after reaching equilibrium in vivo (e.g., upon assuming equilibrium in the total blood volume). When expressed in terms of the amount of the curcuminoid delivered, the patient can receive, for example, an intravenous dose within the range of 0.001 μg/kg-100 μg/kg (e.g., about 0.01, 0.03, 0.05, 0.1, 0.3, 0.5, 1.0, 3.0, 5.0 or 10.0 μg/kg given daily or at such hourly or daily intervals required to maintain a micromolar or sub-micromolar (e.g., a nanomolar or subnanomolar) concentration in the circulation and/or at the site of an ischemic injury). For example, a suitable dose is one that results in a circulating plasma level of the curcuminoid in the micromolar, nanomolar, or picomolar range. Thus, the present methods encompass administration of a curcuminoid at a rate that maintains a circulating level of the curcuminoid at a nanomolar or picomolar concentration (e.g., intravenous administration, oral administration (e.g., from a controlled or sustained release formulation) or topical administration).

For ease of reading, we do not repeat phrases such as “or a pharmaceutically active salt, metabolite or analog thereof” at every occurrence. However, it is to be understood that where a curcuminoid (e.g., curcumin) can be used in the present formulations and in the present methods for treating ischemia (e.g., by reducing the amount of tissue necrosis in the area of the ischemic tissue), a pharmaceutically active salt, metabolite, or analog thereof can also be used.

The methods can be used in veterinary medicine (e.g., to treat pets such as cats and dogs), and in the treatment of human patients. (As the treatment is inexpensive, cost would not impede its use in the treatment of animals.) Regardless of the subject (whether human or non-human), any of the present methods can include a step of identifying the subject (i.e. a subject in need of treatment). Thus, for example, the methods can include a step of determining whether the subject is in need of treatment (e.g., by diagnosis of an ischemic injury, particularly one that is progressing to cause more extensive areas of necrosis).

The curcuminoid can be curcumin, demethoxycurcumin or bisdemethoxycurcumin, or a pharmaceutically acceptable salt thereof, and pharmaceutically active metabolites useful in the formulations and methods of the present invention include tetrahydrocurcumin and dihydrocurcumin.

The ischemic tissue can be the skin, and the ischemic tissue damage can be associated with a burn (e.g., a thermal burn), a diabetic sore, a pressure sore, hypotension, a thrombus, an embolus, or localized exposure to extreme cold (e.g., frostbite or an injury due to a supercooled liquid such as liquid nitrogen). Alternatively or in addition, the ischemic tissue can be a muscle (e.g., the myocardium or a skeletal muscle). Where the ischemic tissue is within the heart, it can be associated with a myocardial infarction or cardiac arrest. Nervous tissue is also sensitive to oxygen deprivation. Thus, the ischemic tissue can be within the brain or spinal cord, and the ischemic tissue damage can be associated with hypotension, a thrombus, an embolus, cardiac arrest, or a traumatic injury.

The curcuminoid can be entrapped in a lipid-based or polymer-based colloid, such as a liposome, nanoparticle, microparticle, or block copolymer micelle, and administered parenterally (e.g., intravenously). The curcuminoid, whether “free” or associated with a colloid, can be in a solution or suspension that includes a buffer (e.g., a phosphate buffered saline) and albumin.

The invention also features methods of treating a patient (e.g., a human patient or a pet) who has a burn to the skin or other externally accessible tissue by administering a curcuminoid, or a pharmaceutically active salt, metabolite, or analog thereof; topically. For example, the curcuminoid can be administered in a topical preparation (e.g., a solution, salve, gel, or ointment) containing a sub-micromolar concentration of the curcuminoid (or a pharmaceutically active salt, metabolite, or analog thereof) or a low dose of the curcuminoid that produces, in the area of the ischemic tissue, sub-micromolar concentrations of the curcuminoid (or the pharmaceutically active salt, metabolite, or analog thereof). For example, the topical preparation can contain a nanomolar (or sub-nanomolar (e.g., picomolar concentration)) of the curcuminoid or the pharmaceutically active salt, metabolite, or analog thereof (e.g., about 1 nM to about 1 pM of the curcuminoid or the pharmaceutically active salt, metabolite, or analog thereof).

The methods described herein can be expressed in terms of “use.” For example, the use of a composition described herein in the preparation of a medicament (e.g., in the preparation of a medicament for the treatment of ischemia).

Also within the scope of the invention are pharmaceutical compositions that include low doses of one or more curcuminoids, or a pharmaceutically active salt, metabolite, or analog thereof. The curcuminoid(s) can be present in the micromolar or sub-micromolar range (e.g., in the nanomolar or subnanomolar (e.g., picomolar or sub-picomolar range)) or in an amount that results, following administration, in sub-micromolar amounts of the curcuminoid in an ischemic tissue within the patient. The curcuminoid can be entrapped in a lipid-based or polymer-based colloid, as described above and further below, and can be formulated as a solution or suspension that includes a buffer (e.g., a phosphate buffered saline) and albumin. Any of the formulations can include a suitable excipient, and the consistency of the formulation can be adjusted in light of the intended route of administration. For example, formulations for intravenous administration can be free flowing liquids, formulations for oral administration can be solids (e.g., tablets or capsules), and formulations for topical administration can be gels, salves, or ointments, optionally preapplied to the wound-contacting surface of a bandage or dressing. These pharmaceutical solutions, suspensions, tablets, capsules, gels, salves, ointments, bandages, and dressings are within the scope of the present invention, provided these compositions contain micromolar or sub-micromolar amounts of the active curcuminoid(s) or an amount that results, following administration, in sub-micromolar amounts of the curcuminoid in an ischemic tissue within a patient. The compositions and methods of the invention sustain sub-micromolar treatment over time (e.g., producing consistently low levels of curcuminoids over the course of hours or days).

The details of one or more embodiments of the invention are set forth in the accompanying drawings and the description below. Other features, objects, and advantages of the invention will be apparent from the description, the studies presented below, and the claims.

### DETAILED DESCRIPTION

The present invention features compositions and methods for treating ischemic tissue damage (which we may sometimes refer to more simply as ischemia) by administering a curcuminoid. The tissue damage can occur in any tissue that is subject to damage by lack of oxygen, whether that damage occurs following a traumatic event (such as a cut or burn) or in the context of a disease or condition where blood vessels are compromised. For example, the ischemia may occur in a patient's skin in the area of a diabetic sore or pressure sore, in the event of hypotension, following a thrombus or embolus, or in response to localized exposure to extreme cold. A thrombus or embolus may also cause ischemia in the heart muscle or brain, leading to a myocardial infarction or cerebrovascular accident, respectively. These conditions specifically, as well as cardiac arrest, and any ischemia generally can be treated according to the present methods.

When ischemia is triggered, the methods described herein can be employed to produce a better long-term outcome than would have been otherwise expected. In other words, “treating” a patient may produce less tissue necrosis than expected in the absence of curcuminoid administration. While the invention is not limited to methods in which treatment occurs by any particular mechanism, we expect the low doses of curcuminoids administered here cause vasodilation and thereby reduce the progression of the ischemic injury. For example, where a patient has received a thermal burn, the administered curcuminoids can reduce the progression of burn injury in the zone of ischemia and inhibit the conversion of partial thickness injuries into full thickness necrosis.

Treatment can begin as soon as an underlying ischemia is detected or suspected, and the present compositions can be administered until the ischemic area is no longer progressing. While the present methods can be applied at various times, treatment will preferably commence soon after the onset of ischemia (e.g., following a burn). For example, treatment can commence within about 1-24 hours (e.g., about 1-2 or 2-4 hours) following the onset of ischemia or the recognition thereof. Accordingly, the treatment may be characterized as a “first line” treatment.

The compositions can be administered to a subject in a variety of ways. For example, the compositions can be administered orally or parenterally (e.g., transdermally or injected (infused) intravenously, subcutaneously, sublingually, intracranially, intramuscularly, intraperitoneally, or intrapulmonarily (i.e., inhaled). Oral formulations are also within the scope of the present invention. Regardless of the formulation and route of administration, the amount of curcumin administered generally will be in an amount sufficient to achieve a circulating concentration, i.e., a plasma concentration, of 10⁻⁹M or less. The treatment regime can vary depending upon various factors typically considered by one of ordinary skill in the art. These factors include the route of administration, the nature of the formulation, the nature of the patient's illness, the subject's size, weight, surface area, age, gender, other drugs being administered to the patient, and the judgment of the attending physician. The compositions can be administered along with or in addition to other treatments for ischemia, for example, immunotherapy, surgery, or drug therapy (e.g., aspirin therapy, statin therapy, oxygen therapy, or antihypertensive therapy).

Our in vivo studies to date have indicated that vasodilation can be induced by lower concentrations of curcumin after preconditioning than before preconditioning. Thus, the vasculature close to the wound may dilate at 100- to 1000-fold lower concentrations than the current EC50 in the peri-wound area following preconditioning. The curcuminoid dosage administered may therefore be higher during an initial or “preconditioning” phase of treatment and lower thereafter. For example, initial dosages of curcumin corresponding to nanomolar application (10⁻⁹ M) may be administered first, with subsequent administration being reduced to the picomolar range (10⁻¹² M). The dosage can therefore be reduced by at least or about 100- to about 1,000-fold as treatment progresses while maintaining improved blood flow in the vicinity of the wound edge. In the in vivo studies described below (in hamsters), the intravenous dose that was found to be efficacious in preventing burn injury was 0.1 to 100 μg/kg, which we believe equates to a circulating plasma concentration of 10⁻⁹ to 10⁻⁶ M. One can readily correlate amounts of curcumin administered by any given route or in any given formulation with circulating plasma concentrations. One can also readily determine in, for example, animal models or human tissue cultures the extent to which the progression of an ischemic area is inhibited by administration of any of the various low dosages and/or formulations described herein.

Curcumin is also known as diferuloylmethane or (E,E)-1,7-bis (4-hydroxy-3- methoxyphenyl)-1,6-heptadiene-3,5,-dione. Curcumin is found naturally in turmeric together with demethoxycurcumin and bisdemethoxycurcum, the structures of which are depicted below.

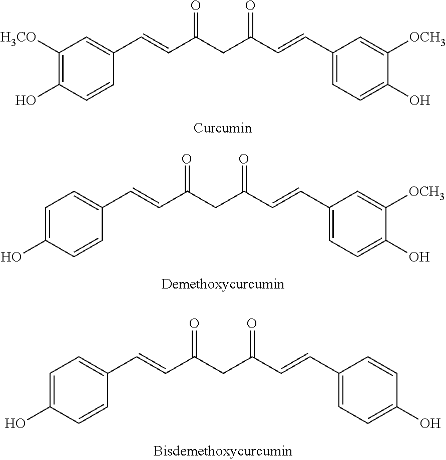 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(O)cc2)ccc1O ; O=C(/C=C/c1ccc(O)cc1)CC(=O)/C=C/c1ccc(O)cc1]

Curcumin may be derived from a natural source, the perennial herb Curcuma longa, which is a member of the Zingiberaceae family. The spice turmeric is extracted from the rhizomes of Curcuma longa and has been used in traditional medicine practiced widely in Indian and Chinese communities. Historically, turmeric is administered most frequently orally or topically.

Curcumin is soluble in ethanol, alkalis, ketones, acetic acid and chloroform, but insoluble in water. Curcumin is therefore lipophilic, and generally readily associates with lipids, including many of those used in colloidal drug-delivery systems. Curcumin can also be formulated as a metal chelate. In the present methods, a curcuminoid can therefore be delivered intravenously or topically in preparations that include an agent that increases the curcuminoid's solubility. These agents include albumin, a wide variety of lipids, and metal chelates.

In addition to the naturally occurring curcuminoid compounds curcumin, demethoxycurcumin, and bis-demethoxycurcumin, which are also referred to as curcumin I, II, and III, respectively, the present methods can be practiced with curcuminoids that, due to their structural similarity to curcumin, exhibit vasoactive effects and are therefore also useful in treating ischemia. These curcuminoids include Ar-tumerone, methylcurcumin, sodium curcuminate, dibenzoylmethane, acetylcurcumin, feruloyl methane, the metabolite tetrahydrocurcumin, 1,7-bis(4-hydroxy-3- methoxyphenyl)-1,6-heptadiene-3,5-dione (curcumin1), 1,7-bis(piperonyl)-1,6- heptadiene-3,5-dione(piperonyl curcumin)1,7-bis(2-hydroxy naphthyl)-1,6-heptadiene- 2,5-dione(2-hydroxyl naphthyl curcumin), 1,1-bis(phenyl)-1,3,8,10-undecatetraene-5,7-dione (cinnamyl curcumin) and the like. Additional curcuminoids useful in the present methods are described in published U.S. patent Application No. 2008/0234320, the entire content of which is hereby incorporated by reference herein. Exemplary curcuminoids described in published U.S. Patent Application No. 2008/0234320 include compounds designated therein as EF1, EF2, EF3, EF4, EF7, EF9, EF19, EF24, EF25, MD6, and MD10 and these compounds are useful in the compositions and methods described herein.

Curcumin analogs, any of which can be readily tested to determine their ability to cause vasodilation at low doses in an area of ischemic tissue, include demethoxy curcumin, bisdemethoxycurcumin, sodium curcuminate, and dibenzoylmethane. In some embodiments, the curcumin analogs are those found in the published U.S. Patent Application No. 2005/0181036, the entire content of which is hereby incorporated by reference herein. Other curcumin analogs (curcuminoids) that may be used include, for example, demethoxycurcumin, bisdemethoxycurcumin, dihydrocurcumin, tetrahydrocurcumin, hexahydrocurcumin, dihydroxytetrahydrocurcumin, Yakuchinone A and Yakuchinone B, and their salts, oxidants, reductants, glycosides and esters thereof. Such analogues are described in published U.S. Patent Application No.: 20030147979; and U.S. Pat. No. 5,891,924 both of which are incorporated in their entirety herein by reference. Derivatives of curcumin and curcumenoids include those derivatives disclosed in published U.S. Patent Application No.: 20020019382, which is hereby incorporated by reference in the present application. Other curcumin analogs are those found in published U.S. Patent Application No.: 2005/0267221, which is hereby incorporated by reference in the present application. Additional exemplary curcumin analogues include but are not limited to (a) ferulic acid, (i.e., 4-hydroxy-3-methoxycinnamic acid; 3,4-methylenedioxy cinnamic acid; and 3,4-dimethoxycinnamic acid); (b) aromatic ketones (i.e., 4-(4- hydroxy-3-methoxyphenyl)-3-buten-2-one; zingerone; -4-(3,4-methylenedioxyphenyly-2- butanone; 4-(p-hydroxyphenyl)-3-buten-2-one; 4-hydroxyvalerophenone; 4- hydroxybenzylactone; 4-hydroxybenzophenone; 1,5-bis(4-dimethylaminophen-yl)-1,4- pentadien-3-one); (c) aromatic diketones (i.e., 6-hydroxydibenzoylmethane) (d) caffeic acid compounds (i.e., 3,4-dihydroxycinnamic acid); (e) cinnamic acid; (f) aromatic carboxylic acids (i.e., 3,4-dihydroxyhydrocinnainic acid; 2-hydroxycinnamic acid; 3-hydroxycinnamic acid and 4-hydroxycinnamic acid); (g) aromatic ketocarboxylic acids (i.e., 4-hydroxyphenylpyruvic acid); and (h) aromatic alcohols (i.e., 4-hydroxyphenethyl alcohol). These analogues and other representative analogues are further described in WO9518606 and WO01040188.

The compound administered may also be an isomer of curcumin, such as a (Z,E) or (Z,Z) isomer. Curcumin metabolites that have vasoactive effects similar to curcumin can also be used to treat ischemia. Known curcumin metabolites include glucoronides of tetrahydrocurcumin and hexahydrocurcumin, and dihydroferulic acid. In certain embodiments, curcumin analogues or metabolites can be formulated as metal chelates, especially copper and zinc chelates. Other appropriate analogs and metabolites of curcumin appropriate for use in the present invention will be apparent to one of ordinary skill in the art.

Pharmaceutically active salts are salts that exhibit one or more of the same biological activities of the parent compound without unacceptable toxicity. Examples of such salts are (a) acid addition salts formed with inorganic acids (e.g., hydrochloric acid, hydrobromic acid, sulfuric acid, phosphoric acid, nitric acid and the like) and organic acids (e.g., acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like) and (b) salts formed from elemental anions such as chlorine, bromine, and iodine.

The pharmaceutically acceptable salts can also be in the form of a pharmaceutically acceptable free base of a corresponding curcumanoid. The free base of the compound may be less soluble than the salt, and therefore better suited for sustained release of the curcuminoid to the target area. Curcuminoids in the target area that have not gone into solution are not available to induce a physiological response, but they can serve as a depot and gradually go into solution.

Curcumin and other curcuminoids are commercially available or can be synthesized by methods known in the art. For example, ChromaDex produces 99.9% pure curcumin I under GMP conditions.

Formulations of one or more curcuminoids suitable for intravenous administration are typically sterile aqueous preparations that are preferably isotonic with the blood of the intended recipient. Such formulations may conveniently be prepared by admixing the compound(s) with water or, more preferably, a buffered solution (e.g., a phosphated buffered solution such as saline or a glycine buffer) and rendering the resulting solution sterile and isotonic with the blood.

We use the terms “pharmaceutically acceptable” (or “pharmacologically acceptable”) to refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

Pharmaceutically acceptable compositions for use in the present methods, including those in which a curcumanoid is entrapped in a colloid, can be prepared according to standard techniques. Generally, a pharmaceutical carrier such as normal saline will be employed. Other suitable carriers include water, buffered water, isotonic aqueous solutions, 0.4% saline, 0.3% aqueous glycine, DMSO, and glycoproteins such as albumin, a lipoprotein, and globulins. The glycoproteins can enhance the stability and/or solubility of the curcuminoid.

While the carrier can be water or can include water, we expect the most effective solutions will include an agent, such as one or more of those discussed herein, that improves the solubility of the curcuminoid (e.g., an agent that reduces or shields its hydrophobic nature). For example, since curcumin binds to albumin, it can be delivered in the presence of albumin. Other agents that facilitate delivery include nanoparticles with a hydrophobic core, micelles, liposomes, and the like. Solubility can also be increased where necessary or desired by the inclusion of an amino sugar (e.g., meglumine, glucosamine, n-acetylglucosamine, sialic acid, and galactosamine). We may use the term “solubilizing agent” to refer to an agent included in the present formulations for the purpose of increasing the solubility of the curcuminoid.

Any of the compositions can be sterilized by conventional sterilization techniques that are well-known in the art, and any of the present methods can employ a step of providing a curcuminoid, sterilizing a composition including it, and administering it to a patient as described herein. Sufficiently small liposomes, for example, can be sterilized using sterile filtration techniques.

The present formulations can include albumin and DMSO at, for example, 0.5-1%. Formulation characteristics that can be modified include, for example, the pH and the osmolality. For example, it may be desired to achieve a formulation that has a pH and osmolality similar to that of human blood or tissues to facilitate the formulation's effectiveness when administered intravenously.

Buffers are useful in the present invention for, among other purposes, manipulation of the total pH of the pharmaceutical formulation (especially desired for parenteral administration). A variety of buffers known in the art can be used in the present formulations, such as various salts of organic or inorganic acids, bases, or amino acids, and including various forms of citrate, phosphate, tartrate, succinate, adipate, maleate, lactate, acetate, bicarbonate, or carbonate ions. Particularly advantageous buffers for use in parenterally administered forms of the presently disclosed compositions in the present invention include sodium or potassium buffers, particularly sodium phosphate. In a preferred embodiment for parenteral dosing, sodium phosphate is employed in a concentration approximating 20 mM to achieve a pH of approximately 7.0. A particularly effective sodium phosphate buffering system comprises sodium phosphate monobasic monohydrate and sodium phosphate dibasic heptahydrate. When this combination of monobasic and dibasic sodium phosphate is used, advantageous concentrations of each are about 0.5 to about 1.5 mg/ml monobasic and about 2.0 to about 4.0 mg/ml dibasic, with preferred concentrations of about 0.9 mg/ml monobasic and about 3.4 mg/ml dibasic phosphate. The pH of the formulation changes according to the amount of buffer used.

In some embodiments, it will also be advantageous to employ surfactants in the presently disclosed formulations, where those surfactants will not be disruptive of the drug-delivery system used. Surfactants or anti-adsorbants that prove useful include polyoxyethylenesorbitans, polyoxyethylenesorbitan monolaurate, polysorbate-20, such as Tween-20TM, polysorbate-80, hydroxycellulose, and genapol. By way of example, when a surfactant is employed in the present invention to produce a parenterally administrable composition, it is advantageous to use it in a concentration of about 0.01 to about 0.5 mg/ml.

Additional useful additives can be readily determined by those of skill in the art, according to particular needs or intended uses of the compositions and formulations. One such particularly useful additional substance is sodium chloride, which is useful for adjusting the osmolality of the formulations to achieve the desired resulting osmolality. Particularly preferred osmolalities for parenteral administration of the disclosed compositions are in the range of about 270 to about 330 mOsm/kg. The optimal osmolality for parenterally administered compositions, particularly injectables, is approximately 300 Osm/kg and achievable by the use of sodium chloride in concentrations of about 6.5 to about 7.5 mg/ml with a sodium chloride concentration of about 7.0 mg/ml being particularly effective.

Curcumin-containing liposomes or curcumin-containing colloidal drug-delivery vehicles can be stored as a lyophilized powder under aseptic conditions and combined with a sterile aqueous solution prior to administration. The aqueous solution used to resuspend the liposomes can contain pharmaceutically acceptable auxiliary substances as required to approximate physical conditions, such as pH adjusting and buffering agents, tonicity adjusting agents and the like, as discussed above.

In other embodiments, the curcumin-containing liposomes or curcumin-containing colloidal drug-delivery vehicle can be stored as a suspension, preferably an aqueous suspension, prior to administration. In certain embodiments, the solution used for storage of liposomes or colloidal drug carrier suspensions will include lipid-protective agents which protect lipids against free-radical and lipid-peroxidative damage on storage. Suitable protective compounds include free-radical quenchers such as alpha-tocopherol and water-soluble iron-specific chelators, such as ferrioxamine.

Formulations useful in delivering curcuminoids at the dosages described herein are described in U.S. patent Application No. 2006/0067998, the entire content of which is hereby incorporated by reference in its entirety. Useful compounds for the formation of liposomes are well known in the art and include synthetic vesicle-forming lipids and naturally-occurring vesicle-forming lipids, including the sphingolipids, ether lipids, sterols, phospholipids, particularly the phosphoglycerides, and the glycolipids, such as the cerebrosides and gangliosides. Phosphoglycerides include phospholipids such as phosphatidylcholine, phosphatidylethanolamine, phosphatidic acid, phosphatidylinositol, phosphatidylserine phosphatidylglycerol and diphosphatidylglycerol (cardiolipin), where the two hydrocarbon chains are typically between about 14-22 carbon atoms in length, and have varying degrees of unsaturation.

Exemplary phosphatidylcholines include dilauroyl phophatidylcholine, dimyristoylphophatidylcholine, dipalmitoylphophatidylcholine, distearoylphophatidyl-choline, diarachidoylphophatidylcholine, dioleoylphophatidylcholine, dilinoleoyl-phophatidylcholine, dierucoylphophatidylcholine, palmitoyl-oleoyl-phophatidylcholine, egg phosphatidylcholine, myristoyl-palmitoylphosphatidylcholine, palmitoyl-myristoyl-phosphatidylcholine, myristoyl-stearoylphosphatidylcholine, palmitoyl-stearoyl-phosphatidylcholine, stearoyl-palmitoylphosphatidylcholine, stearoyl-oleoyl-phosphatidylcholine, stearoyl-linoleoylphosphatidylcholine and palmitoyl-linoleoyl-phosphatidylcholine. Assymetric phosphatidylcholines are referred to as 1-acyl, 2-acyl- sn-glycero-3-phosphocholines, wherein the acyl groups are different from each other. Symmetric phosphatidylcholines are referred to as 1,2-diacyl-sn-glycero-3-phosphocholines.

The curcuminoids of the invention may also be formulated in “caged” phospholipids, i.e., aminophospholipids that are pH-sensitive such that the caging groups groups are cleaved in the intracelllular environment and the contents of liposome are released; caged phospholipids are described in U.S. Pat. No. 5,972,380, which is incorporated by reference herein. Exemplary phosphatidylethanolamines include dimyristoyl-phosphatidylethanolamine, dipalmitoyl-phosphatidylethanolamine, distearoyl-phosphatidylethanolamine, dioleoyl-phosphatidylethanolamine and egg phosphatidylethanolamine. Exemplary phosphatidic acids include dimyristoyl phosphatidic acid, dipalmitoyl phosphatidic acid and dioleoyl phosphatidic acid. Exemplary phosphatidylserines include dimyristoyl phosphatidylserine, dipalmitoyl phosphatidylserine, dioleoylphosphatidylserine, distearoyl phosphatidylserine, palmitoyl-oleylphosphatidylserine and brain phosphatidylserine. Exemplary phosphatidylglycerols include dilauryloylphosphatidylglycerol, dipalmitoylphosphatidylglycerol, distearoylphosphatidylglycerol, dioleoyl-phosphatidylglycerol, dimyristoylphosphatidylglycerol, palmitoyl-oleoyl-phosphatidylglycerol and egg phosphatidylglycerol. Suitable sphingomyelins might include brain sphingomyelin, egg sphingomyelin, dipalmitoyl sphingomyelin, and distearoyl sphingomyelin Other suitable lipids include glycolipids, sphingolipids, ether lipids, glycolipids such as the cerebrosides and gangliosides, and sterols, such as cholesterol or ergosterol. Additional lipids suitable for use in liposomes are known to persons of skill in the art and are cited in a variety of sources, such as 1998 McCutcheon's Detergents and Emulsifiers, 1998 McCutcheon's Functional Materials, both published by McCutcheon Publishing Co., New Jersey, and the Avanti Polar Lipids, Inc. Catalog, which are herein incorporated by reference.

Suitable lipids for use in the present invention will have sufficient long-term stability to achieve an adequate shelf-life. Factors affecting lipid stability are well-known to those of skill in the art and include factors such as the source (e.g. synthetic or tissue-derived), degree of saturation and method of storage of the lipid.

The formation and use of liposomes is generally known to those of skill in the art, as described in, e.g. Liposome Technology, Vols. 1, 2 and 3, Gregory Gregoriadis, ed., CRC Press, Inc; Liposomes: Rational Design, Andrew S. Janoff, ed., Marcel Dekker, Inc.; Medical Applications of Liposomes, D. D. Lasic and D. Papahadjopoulos, eds., Elsevier Press; Bioconjugate Techniques, by Greg T. Hermanson, Academic Press; and Pharmaceutical Manufacturing of Liposomes, by Francis J. Martin, in Specialized Drug Delivery Systems (Praveen Tyle, Ed.), Marcel Dekker, Inc., all of which are herein incorporated by reference.

The present methods for treating ischemia are carried out by administering administering to the patient a curcuminoid or a pharmaceutically active salt or metabolite thereof, wherein the curcuminoid is administered at a dose within the range of 0.01 μg/kg -100 μg/kg.

The amount and frequency of administration of the compositions can vary depending on, for example, what is being administered, the state of the patient, and the manner of administration. In therapeutic applications, compositions can be administered to a patient suffering from ischemia in an amount sufficient to relieve or least partially relieve the symptoms of ischemia and its complications. The dosage is likely to depend on such variables as the type and extent of progression of the ischemia, the severity of the ischemia, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of ischemia or slowing its progression.

The amount of curcuminoid per dose can vary. For example, a subject can receive from about 0.01 μg/kg to about 100 μg/kg., e.g., about 0.01, 0.02, 0.05. 0.75, 1.0, 1.5, 2.0. 5.0. 10.0. 15.0, 20.0, 25.0, 30.0, 40.0, 50.0, 60.0, 70.0, 80.0, 90.0 or 100.0 μg/kg. Generally, we administer a curcuminoid in an amount such that the circulating concentration does not exceed a nanomolar concentration (e.g., 10⁻⁶ M). For example, the compositions and methods of the invention can achieve circulating levels of the curcuminoid or levels of the curcumoid in the vasculature or tissue at the site of ischemia of 10⁻⁶ to 10⁻⁹M or less. Thus, the curcuminoid can be administered in an amount such that the circulating concentration of the curcuminoid is, for example, 0.001 nM. 0.005 nM, 0.010 nM, 0.050 nM, 0.100 nM, 0.500 nM, 1.0 nM, 5.0 nM, 10 nM, 50 nM, 100 nM, 500 nM or 1000 nM. Exemplary dosages can produce a circulating concentration of the curcumoid in the subject of up to or up to about 1 nM, 10 nM or 100 nM.

The frequency of treatment may also vary. Preferably, the levels of curcuminoid in the circulation or in the vasculature or tissue at the site of ischemia are maintained fairly consistently at the low levels described herein. Preferably, the tissue is exposed to these low levels of a curcuminoid as soon as possible after the onset of an injury or event that leads to ischemia (e.g., a thrombus or embolus). The subject can be treated one or more times per day (e.g., once, twice, three, four or more times) or every so-many hours (e.g., about every 2, 4, 6, 8, 12, or 24 hours). The time course of treatment may be of varying duration, for example, for two, three, four, five, six, seven, eight, nine, ten or more days. For example, the treatment can be twice a day for three days, twice a day for seven days, twice a day for ten days. While our expectation is that the treatment will continue as the patient's tissues go through a healing and/or remodeling process, treatment cycles can be repeated at intervals. For example treatment can be repeated weekly, bimonthly or monthly, and the periods of treatment can be separated by periods in which no treatment is given. The treatment can be a single treatment or can last as long as the life span of the subject (e.g., many years).

Method of the invention are applicable to any of a wide range of medical conditions which have as their underlying feature a persistent reduction of or partial or complete blockage of blood flow to a tissue or organ. Thus, the methods are applicable to treatment of chronic tissue ischemia associated with a disorder, with a trauma or an environmental stress. The reduction in blood flow to a tissue can be, for example, the result of a progressive blockage of an artery due to hardening and/or loss of elasticity due to an atheromatous plaque or the presence of a clot. Reduction of blood flow to a tissue can also be the result of an environmental insult, for example, a traumatic injury or surgical procedure that interrupts the blood flow to a tissue or organ. Typically, the oxygen tension of a wound quickly and progressively decreases with the development of varying degrees of hypoxia throughout the wound region. Environmental conditions that induce hypoxia are also within the scope of the invention.

The methods of the invention are applicable to ischemia associated with a trauma, for example, a traumatic injury such as a burn, wound, laceration, contusion, bone fracture or chronic infection. Also encompassed by the invention are tissue injuries sustained as part of any surgical procedure, for example, endarterectomy. Procedures involving tissue or organ transplantation are within the scope of the invention. Examples include vascular bypass grafts, heart, liver, lung, pancreatic islet cell transplantation as well as transplantation of tissues generated ex vivo for implantation in a host. The methods of the invention are also useful for treating an ischemic condition brought about by exposure to an environmental insult, for example, chronic exposure to hypoxic conditions, for example, high altitude, or sustained aerobic exertion. Disorders encompassed by the invention include ischemia associated with or brought about by cardiovascular disease, peripheral artery disease, arteriosclerosis, atherosclerotic cardiovascular disease, myocardial infarction, critical limb ischemic disease, stroke, acute coronary syndrome, intermittent claudication, diabetes, including type 1 and type 2 diabetes, skin ulcers, peripheral neuropathy, inflammatory bowel disease, ulcerative colitis, Crohn's disease, intestinal ischemia, and chronic mesenteric ischemia.

The methods provided herein are applicable to any of a wide range of tissue types including, for example, muscle, smooth muscle, skeletal muscle, cardiac muscle, neuronal tissue, skin, mesechymal tissue, connective tissue, gastrointestinal tissue or bone. Soft tissue, such as epithelial tissue, for example, simple squamous epithelia, stratified squamous epithelia, cuboidal epithelia, or columnar epithelia, loose connective tissue (also known as areolar connective tissue), fibrous connective tissue, such as tendons, which attach muscles to bone, and ligaments, which join bones together at the joints.

The methods of the invention can include the steps of identifying a subject (e.g., a human patient) who is experiencing or is likely to experience tissue ischemia. Since ischemia can result from a wide range of medical conditions all of which have as their underlying feature a persistent reduction of or partial or complete blockage of blood flow to a tissue, the specific signs and symptoms will vary depending upon factor or factors responsible for the reduction of blood flow.

For example, a burn injury can result from exposure to heat, electricity, chemicals, light, radiation, or friction. Burn injury induces skin loss in two stages: there is an immediate necrosis resulting from direct dissipation of thermal energy and a delayed necrosis resulting from the loss of blood flow to the dermis surrounding the burn. A burn injury becomes deeper and larger with the loss of this surrounding tissue. The delayed necrosis results from vascular occlusion that results in local ischemia. If blood flow returns to these occluded vessels soon after trauma, the tissue will survive and the expansion of tissue loss will not occur. Preventing or reversing vascular occlusion and the reestablishment of blood flow can reduce the volume of tissue loss by secondary ischemia.

Burn injuries are classified as first, second, third, fourth and fifth degree. Symptoms include: 1) first-degree burns, which involve only the epidermis: redness (erythema), a white plaque and minor pain at the site of injury; 2) second-degree burns, which involve the superficial (papillary) dermis and may also involve the deep (reticular) dermis: erythema with superficial blistering of the skin, and pain depending on the level of nerve involvement: 3) third-degree burns, which involve loss of the epidermis with damage to the subcutaneous tissue: charring and extreme damage of the epidermis, and sometimes hard eschar will be present; these burns are not painful, as the damaged nerves are unable to transmit pain signals; however, all third-degree burns are surrounded by first and second-degree burns, which are painful; 4) fourth-degree burns occur when heat damage destroys the dermis and muscle is affected; 5) fifth-degree burns occur when all the skin and subcutaneous tissues are destroyed, exposing muscle. These burns can be fatal due to breaches of major arteries and veins; 6) sixth-degree burns occur when heat destroys the muscles, charring and exposing the bone.

The methods of the invention can be administered in combination with other standard treatments for burn injuries, for example, standard first aid treatments such as cooling or bandaging; surgical remedies such as excision and/or tissue grafting; standard wound management techniques; therapeutics such as analgesics, antibiotics and biologics, for example, growth factors and other treatments such as administration of intravenous fluids or hyperbaric oxygenation.

Symptoms of tissue ischemia in peripheral artery disease (PAD), a form of peripheral vascular disease in which there is partial or total blockage of an artery, usually due to atherosclerosis in a vessel or vessels leading to a leg or arm, can include intermittent claudication, that is, fatigue, cramping, and pain in the hip, buttock, thigh, knee, shin, or upper foot during exertion that goes away with rest, claudication during rest, numbness, tingling, or coldness in the lower legs or feet, neuropathy, or defective tissue wound healing. PAD in the lower limb is often associated with diabetes, particularly type 2 diabetes. Arm artery disease is usually not due to atherosclerosis but to other conditions such as an autoimmune disease, a blood clot, radiation therapy, Raynaud's disease, repetitive motion, and trauma. Common symptoms when the arm is in motion include discomfort, heaviness, tiredness, cramping and finger pain. PAD can be diagnosed by performing one or more diagnostic tests including, for example, an ankle brachial index (ABI) test, angiography, ultrasound, or MRI analysis.

Myocardial ischemia can have few or no symptoms, although typically, it is associated with a symptoms such as angina, pain, fatigue elevated blood pressure. Diagnostic tests for myocardial ischemia include: angiography, resting, exercise, or ambulatory electrocardiograms; scintigraphic studies (radioactive heart scans); echocardiography; coronary angiography; and, rarely, positron emission tomography.

The method of the invention can also be used in conjunction with other remedies known in the art that are used to treat ischemia including, drug therapy, surgery, anti-inflammatory agents, antibodies, exercise, or lifestyle changes. The choice of specific treatment may vary and will depend upon the severity of the ischemia, the subject's general health and the judgment of the attending clinician. The present compositions can also be formulated in combination with one or more additional active ingredients, which can include any pharmaceutical agent such antihypertensives, anti-diabetic agents, statins, anti-platelet agents (clopidogrel and cilostazol), antibodies, immune suppressants, anti-inflammatory agents, antibiotics, chemotherapeutics, and the like. The curcuminoid treatments of the present invention can be administered in combination with other treatments for ischemia (e.g., oxygen therapy or conventional vasodilatory compounds). Curcuminoids, formulated as described herein, can also be administered together with α-adrenergic antagonists, which may enhance vasodilation in the region of the ischemic injury. Some such antagonists are known in the art and include phentolamine (e.g., phentolamine mesylate).

As ischemia often occurs in the context of an emergency situation, the present formulations can be included within kits that are packaged to allow rapid administration of the formulations. For example, a kit may include a curcuminoid formulation in an intravenous bag that can be readily punctured and assembled to provide an intravenous infusion to the patient. As noted, semi-solid formulations may also be applied to the wound-contacting portion of a bandage or dressing.

### EXAMPLES

While the present compositions and methods are not limited to those that confer a therapeutic benefit by any particular mechanism, we speculated that curcumin limits the progression of injury from an ischemic site by optimizing microvascular nutrient blood flow in the vicinity of the injury (e.g., the burn). To investigate this, we first examined the microvascular response to locally applied curcumin in the mucosal region of the hamster cheek pouch tissue using intravital microscopy.

### Example 1

### Application of Curcumin to Hamster Cheek Pouch Tissue

The cheek pouch tissue was exteriorized in anesthetized male, adult hamsters (120±12 g, 116±34 days, N=60; pentobarbital at 70 mg/kg). The observation site was the feed of a terminal arteriolar network (baseline diameter, 8±2 μm) and the arcade arteriole (22±1 μm) that supplied it; the terminal arteriolar network controls nutrient flow to the capillaries (one terminal feed arteriole providing nutrient flow to 3-5 terminal branches). We examined the mucosal region only. In the present study curcumin was applied via micropipette to the entrance to the terminal network as the terminal arteriole arose from the arcade, thus defining the response to curcumin for two classes of blood vessels: conduit arcade arterioles and nutritive terminal arterioles (directly feeding capillaries). The curcumin (10⁻¹² to 10⁻⁴ M) was applied in increasing doses using micropipette delivery for 60 seconds. (A control tissue bath solution of bicarbonate buffered saline was flowed continuously over the tissue at 5 ml/minute.) Adenosine (10⁻⁴mol/L) and phenylephrine (10⁻⁴ mol/L) were dripped (10⁻⁴ L) onto the tissue and used to confirm dilator and constrictor tone, respectively. Thirty minutes later, microvascular responses (diameter change) were obtained according to one of the following protocols.

Protocol 1—Locally applied curcumin: Curcumin obtained from Chromadex (Irvine, Calif.) was previously tested for purity; this ethanol extraction process followed by preparative HPLC yielded curcumin I (>99%) as demonstrated by mass spectroscopy. The purified curcumin (10⁻² mol/L) was stored at −80 ° C. in ethanol until used, and then diluted in control suffusate (10⁻¹² mol/L-10⁻⁴ mol/L, n=7). In each animal, two to three networks were tested (minimum of 500 μm apart), performing the complete concentration response at each site. This distance assured independent observations in this tissue with curcumin, likewise, there was no difference in responses between sites. The highest dose of curcumin tested (10⁻⁴ mol/L) contained 1% ethanol. An ethanol dose response was repeated here using 0.0001-1% ethanol, encompassing the range of 10⁻⁸ to 10⁻⁴mol/L curcumin (n=3). Curcumin, or ethanol alone, was applied for 60 seconds via micropipette to the junction where the arteriolar terminal feed arose from the arcade, exposing both vessel segments.

Protocol 2—Suffusate applied antagonists: Only one antagonist was tested per animal, and two to three sites that were more than 500 microns apart were tested per animal. Phentolamine (α-adrenergic receptor antagonist, 10⁻⁵mol/L, n=6), propranolol (β-adrenergic receptor antagonist, 10⁻⁵mol/L, n=6), atropine (muscarinic receptor antagonist, 10⁻⁷ mol/L, n=7), or N^{ω}-nitro-L-arginine (LNNA, nitric oxide synthase antagonist 10⁻⁵ mol/L, n=5; and 10⁻⁴ mol/L, n=7) were added to the flowing control suffusate for 5 minutes before and then continuously while Protocol 1 was performed. In five additional animals, phentolamine and propranolol were applied together. Blockade was confirmed with phenylephrine (a-adrenergic receptor agonist, 10⁻⁵ mol/L), isoproterenol (adrenergic receptor agonist, 10⁻⁵ mol/L), acetylcholine (muscarinic receptor agonist, 10⁻⁴ mol/L) and nitroprusside (cGMP mediated dilation agonist, 10⁻⁴ mol/L).

The sympathetic nerve toxin, 6-hydroxy dopamine (6-OHDA, 10⁻³ mol/L, n=5) was applied to a stationary tissue bath. Suffusate flow was stopped, and bone wax was used to create a pool encircling the cheek pouch tissue. The neurotoxin was added to the pool for 20 minutes. Then, control suffusate flow was returned, and Protocol 1 was performed.

Protocol 3—Micropipette applied antagonists: Only one antagonist was tested per animal, and two to three sites were tested per animal. PD142893 (endothelim receptor A and B antagonist, 10⁻⁵ mol/L, n=4), was applied to the observation site via micropipette for 5 minutes prior to and then continuously during curcumin exposure. Curcumin (10⁻¹²-10⁻⁴ mol/L) was applied in increasing concentration, as per Protocol 1. Rp-8Br-cGMPS (cBMP antagonist, 10⁻⁴ mol/L), or Rp-8Br-cAMPS (cAMP antagonist, 10⁻⁴ mol/L), separately and together at separate sites within the same animal (n=5), were applied to the observation site via micropipette for 5 minutes prior to and then continuously during curcumin exposure to only 10⁻⁸ or 10⁻⁶ mol/l curcumin. Blockade was confirmed with nitroprusside (cGMP mediated dilation agonist, 10⁻⁴ mol/L) and adenosine (cAMP mediated dilation agonist, 10⁻⁴ mol/L).

In both the arcade and feed, a biphasic response was observed over time and with increasing doses. There was an initial dilation, predominant at low doses, followed by constriction, predominant at high doses. The fitted logEC50 and peak responses were similar for the arcade and feed. The logEC50 for dilation was −9.5±0.3, with peak dilation of +40±8%. For constriction, logEC50 was −8.4±0.4, peak constriction of −15±5%. Simultaneous atropine (a muscarinic antagonist, 10⁻⁷ M) or PD142893 (an endothelin antagonist, 10⁻⁵ M) had no effect on the curcumin response. Propranolol (a beta-adrenergic antagonist, 10⁻⁵ M) removed the dilation component, enhancing constriction to curcumin (logEC50 −11±0.6; peak −30 ±7%). Phentolamine (an alpha-adrenergic antagonist, 10⁻⁵ M) removed the constrictor component, enhancing dilation to curcumin (logEC50, −10±0.4; peak +34±9 arcade, +65 +10 feed). As the mucosal region of the hamster cheek pouch has only sensory nerves (no sympathetic nerve endings), these finding suggest that curcumin is acting directly through the alpha and beta adrenergic receptors. In conclusion, these findings suggest that curcumin modulates arteriolar diameter specifically via the adrenergic receptors in a dose sensitive manner.

Given these findings, we next tested the effect of curcumin on arteriolar diameter during induced endothelial dysfunction (associated with extensive inflammatory damage), and after microvascular preconditioning (associated with minor oxidative exposure). Endothelial dysfunction or microvascular preconditioning are each commonly associated with inflammatory states; they are associated with injuries, including neurogenic inflammation, ischemia/reperfusion, and other oxidative damage. Endothelial dysfunction was deliberately induced in a standard manner by adding nitro-arginine to the tissue bath of the hamster cheek pouch preparation of anesthetized hamsters. Overall, the dilation component was potentiated and constriction was attenuated. This response is consistent with the known pharmacology of the beta-adrenergic receptor action on the endothelial vs. smooth muscle cells. Further, these findings suggest that in the face of endothelial dysfunction, curcumin preferentially causes dilation in the vicinity of the worst part of the inflamed tissue.

Secondly, we tested the local response to curcumin after microvascular preconditioning. Preconditioning involves a small oxidative insult that enhances dilation through cGMP mechanisms, and decreases dilation through cAMP mechanisms (Am. Physiol. Hear Circ. Physiol. 290:H264-H271, 2006; Microcirculation, 14:739-751, 2007). Preconditioned tissue is not directly damaged but, instead, within this context, it is tissue that is compromised (biochemically altered) by association (or proximity) to the damaged tissue. After deliberate microvascular preconditioning, the local response to curcumin was predominantly constriction, consistent with a lack of cAMP mediated dilation (e.g., beta-adrenergic receptor system), leaving only the alpha-adrenergic mediated constriction. This suggests that tissue that is preconditioned, yet not damaged to the extent of endothelial dysfunction, would show a decreased nutrient flow, which would divert nutrient flow to the nearby regions where the microvascular networks are still intact but greater damage has occurred. Together, these results suggest that the physiologic mechanism by which curcumin prevents burn injury is by diverting nutrient flow to the worst part of the damaged tissue.

### Example 2

### The Effect of Curcumin in an in vivo Burn Model

In this study, we determined the effects of curcumin on burn progression in the rat hot comb model. Two initial studies were performed with crude curcumin (Sigma, Inc.) and demonstrated inhibition of burn injury progression. Pure curcumin (99.9% as determined by HPLC and mass spectroscopy analysis) produced under GMP was acquired from ChromaDex, Inc for the studies described below.

Animals were randomized to receive one of six intravenous doses of curcumin (0.3, 1.0, 3.0, 10, 30, or 100 μg/kg) in 1 ml of phosphate buffered saline (PBS) or PBS alone (buffer control) administered via the tail vein at 1 and 24 hours after injury. Wounds were observed at 2, 5, and 7 days after injury for visual evidence of necrosis in the unburned interspaces by an observer blinded to the protocol (macroscopic evaluation). Full-thickness biopsies from the interspaces 7 days after injury were evaluated for evidence of necrosis after H&E staining. The percentages of interspaces that progressed to necrosis were compared with chi-squared (χ2) tests. At the seventh day, the number of interspaces that processed to full thickness necrosis was 67% for control, 58, 53, 37, 63, 53, and 26% for 0.1, 0.3, 1, 3, 10, 30 and 100 μg/kg curcumin respectively as determined by histologic analysis. Similar results were obtained for the macroscopic evaluation at day 2, 5, and 7. Interestingly, curcumin showed two peaks of significant activity at 3μg/kg and 100 μg/kg that inhibited burn progression. When compared to control, the 3 μg/kg and 100 μg/kg curcumin treatment groups had significantly less progression to necrosis (p<0.01) for both macroscopic evaluation and histologic analysis. The same experiment was repeated a second time and gave essentially the same results. These findings indicated that the treatment with intravenous curcumin can significantly reduce the progression of burn injury in a rat comb burn model.

### Example 3

### Various Effects of Curcumin

We examined the effect of curcumin on terminal arteriole diameter in a 60 second time-course using the hamster check pouch model described above. Curcumin was continuously applied according to the micropette method, described above, at both 10⁻¹⁰M and 10⁻⁶M concentrations. Over the 60 second exposure time, the low nanomolar concentration, i.e., 10⁻¹⁰M, of curcumin induced a sustained dilation. At the micromolar concentration, i.e., 10⁻⁶M, initial vasodilation at 20 seconds was followed by vasoconstriction that peaked at 60 seconds.

We then performed a dose-response analysis of the effect of curcumin on arteriole diameter in both arcade arterioles and terminal arterioles using the hamster cheek pouch model as described above; curcumin was applied in concentrations ranging from 10⁻¹³M to 10⁻³M according to the Protocol 1. Data were collected at both 20 seconds (the “Early” timepoint) and at 60 seconds (the “Late” timepoint) for each concentration and tissue. Corresponding EC50 and maximal values were determined. Peak dilation was significantly greater for terminal arterioles than for arcade arterioles.

Two experiments were performed to explore whether the biphasic nature of the vasomotor response to curcumin was attributable to vehicle (ethanol) or to a possible cytotoxic effect of curcumin over the course of the experiments. The effect of various concentrations of ethanol, i.e., concentrations that corresponded to the concentration of ethanol in the curcumin samples, was assayed according to the method used in the dose-response analysis above. Vehicle (ethanol) alone caused significant constriction at 0.1% and 1%, but only the highest concentration of ethanol (1%) yielded a constriction that could not be distinguished from constriction obtained in the presence of curcumin.

The potential role of cytotoxicity was assessed by measuring the baseline diameter recovery and tone following curcumin exposure. Both constrictor and dilatory (cGMP and cAMP mediated) responses were unchanged before and after repeated exposure to curcumin. These data showed that curcumin stimulated a recoverable dose and time dependent dilation/constriction response in hamster cheek pouch arterioles. This response was robust and sustained at picomolar to low nanomolar levels and recoverable even after repeated 60 second exposures.

### Example 4

### The Effect of Adrenergic Blockade on Curcumin-induced Vasodilation and Vasoconstriction

We examined the role of adrenergic receptors on the curcumin-induced effects on arteriole diameter using the hamster check pouch model described above. The β-adrenergic antagonist, propanolol and the α-adrenergic antagonist, phentolamine, were applied according to the method described in Protocol 2. Early (20 seconds) and Late (60 seconds) diameter changes in response to curcumin in the presence of 10⁻⁵M propranolol or 10⁻⁵M phentolamine for the terminal arteriole were determined. The experiment in which the antagonists were applied separately indicated that adrenergic blockade suppressed curcumin-induced effects on arteriole diameter. The dilation response to curcumin in the terminal arteriole was abolished by the β-adrenergic antagonist, propanolol and the constriction response abolished by α-adrenergic antagonist, phentolamine. The EC50 and maximal values for both the terminal arteriole and arcading arteriole were determined. Blockade was confirmed with phenylephrine (α-adrenergic receptor agonist, 10⁻⁵ mol/L) and isoproterenol (adrenergic receptor agonist, 10⁻⁵ mol/L). We also assayed the effect of co-application of the two antagonists in the presence of three concentrations of curcumin: 10⁻¹⁰ M, 10⁻⁸M and 10⁻⁶M in both terminal and arcade arterioles. Propranolol and phentolamine applied together blocked all response to curcumin. Taken together, out data indicate that curcumin acted, at least in part, through direct action on the vascular wall.

### Example 5

### The Effect of Muscarinic Receptor Blockade, Endothelin Receptor Blockade, Nitric Oxide Blockade, and Cyclic Nucleotide Blockade on Curcumin-induced Vasomodulation

We examined the role of muscarinic and endothelin receptors on the curcumin-induced effects on arteriole diameter using the hamster check pouch model described above. The muscarinic antagonist, atropine and the endothelin antagonist, PD142893, were applied according to the method described in Protocol 2. Atropine (muscarinic antagonist) or PD142893 (endothelin antagonist) each diminished the maximal dilation and enhanced the maximal constriction to curcumin, yet had no effect on baseline diameters. Blockade was confirmed with acetylcholine (muscarinic receptor agonist, 10⁻⁴ mol/L) and endothelin (10⁻⁸M).

We examined the role of nitric oxide on the curcumin-induced effects on arteriole diameter by blocking endogenous nitric oxide (NO) formation with the nitric oxide synthase antagonist, N^{ω}-nitro-L-arginine (LNNA). LNNA according to the method described in Protocol 2. The Early (20 s) and Late (60 s) terminal arteriole diameter changes in response to curcumin in the presence of 10⁻⁵M LNNA and 10⁻⁴M LNNA were determined. The EC50 and maximal values for both the terminal arteriole and arcading arteriole were also determined. Blockade was confirmed with acetylcholine.

LNNA partially inhibited curcumin-induced vasodilation. At 10⁻⁵M LNNA, curcumin-induced dilation was attenuated in both terminal feed arterioles; however, a significant dilation remained at the 10⁻¹² to 10⁻¹⁰ M curcumin concentrations. A similar pattern was observed in arcade arterioles. At 10⁻⁴M LNNA, significant curcumin-induced dilation remained at the lower concentrations of curcumin, e.g., 10⁻¹⁰ though 10⁻⁷ M. These results suggested that curcumin-induced dilation was mediated by NO at least in the nanomolar and micromolar range, but other mechanism(s) may have contributed to the microcirculation response in the picomolar range.

We analyzed the mechanism of curcumin-induced vasoactivity by targeting specific cyclic nucleotides. The β-Ad receptors may be present on endothelial cells, where they induced a NO, cGMP mediated dilation. Alternatively, or in addition, β-Ad receptors may be present on vascular smooth muscle cells where they induced a cAMP mediated dilation. We evaluated the effect of direct blockade of cGMP and cAMP using the Rp isomers, Rp-8-br-cGMPS and Rp-8-br-cAMPS according to the method described in Protocol 3. We measured the Early (20 s) and Late (60 s) diameter changes in response to both 10⁻⁸M and 10⁻⁶M curcumin in the presence of 10⁻⁵M Rp-8-br-cGMPS (to block cGMP) or cAMP (Rp-8-br-cAMPS (to block cAMP) for both terminal and arcade arterioles. Blockade was confirmed with adenosine (10⁻⁴M) and nitroprusside (cGMP mediated dilation agonist, 10⁻⁴ mol/L).

Blocking cAMP significantly suppressed curcumin-induced dilation to at 10⁻⁶M for the arcade, but not terminal arterioles. Blocking cAMP also attenuated curcumin-induced constriction to 10⁻⁸M in arcade, but not terminal arterioles. In contrast, blocking cGMP prevented all curcumin-induced dilation in both the terminal and arcade arterioles. Taken together, these data suggested 1) a significant role for cAMP in curcumin-induced dilation for the larger arcade arterioles; and 2) that the curcumin-induced dilation appeared to require cGMP for both classes of vessels.

Antagonist blockade of curcumin-induced vasodilation and/or vasoconstriction was confirmed with phenylephrine (a-adrenergic receptor agonist, 10⁻⁵ mol/L); isoproterenol (adrenergic receptor agonist, 10⁻⁵ mol/L); acetylcholine (muscarinic receptor agonist,10⁻⁴ mol/L); nitroprusside (cGMP mediated dilation agonist, 10⁻⁴ mol/L) and adenosine (cAMP mediated dilation agonist, 10⁻⁴ mol/L) and adenosine (10⁻⁴ M) according to the methods described above.

What is claimed is:

1. A method of treating a patient for ischemic tissue damage, the method comprising administering to the patient a curcuminoid or a pharmaceutically active metabolite or analog thereof, wherein the curcuminoid is administered intravenously at a dose within the range of 0.01 μg/kg-100 μg/kg.

2. (canceled)

3. The method of claim 1, wherein the curcuminoid is curcumin.

4. The method of claim 1, wherein the curcuminoid is demethoxycurcumin or bisdemethoxycurcumin.

5. The method of claim 1, wherein the pharmaceutically active metabolite is tetrahydrocurcumin or dihydrocurcumin.

6. The method of claim 1, wherein the ischemic tissue is the skin.

7. The method of claim 6, wherein the ischemic tissue damage is associated with a thermal burn.

8.-10. (canceled)

11. The method of claim 1, wherein the ischemic tissue is a muscle.

12. The method of claim 11, wherein the muscle is the myocardium and the ischemic tissue damage is associated with a myocardial infarction or cardiac arrest.

13.-14. (canceled)

15. The method claim 1 wherein the ischemic tissue is within the brain or spinal cord.

16.-18. (canceled)

19. The method of claim 1, wherein the curcuminoid is entrapped in a lipid-based or polymer-based colloid.

20. -22. (canceled)

23. A method of treating a patient for ischemic tissue damage, the method comprising administering to the patient a curcuminoid or a pharmaceutically active salt or metabolite thereof, wherein the curcuminoid is administered intravenously at a rate that maintains a circulating level of the curcuminoid at a nanomolar or picomolar concentration.

24. (canceled)

25. The method of claim 23, wherein the curcuminoid is curcumin, demthoxycurcumin or bisdemethoxycurcumin.

26. (canceled)

27. The method of claim 23, wherein the pharmaceutically active metabolite is tetrahydrocurcumin or dihydrocurcumin.

28. The method of claim 23, wherein the ischemic tissue is the skin.

29. The method of claim 28, wherein the ischemic tissue damage is associated with a thermal burn.

30. (canceled)

31. The method of any of claim 23, wherein the ischemic tissue is a muscle.

32. (canceled)

33. The method of claim 23, wherein the ischemic tissue is within the brain or spinal cord.

34. (canceled)

35. The method of claim 23, wherein the curcuminoid is entrapped in a lipid-based or polymer-based colloid.

36.-40. (canceled)

41. A method of treating a patient who has a burn to the skin or other externally accessible tissue, the method comprising administering to the patient a curcuminoid or a pharmaceutically active salt, metabolite, or analog thereof, wherein the curcuminoid is administered in a topical preparation containing a sub-micromolar concentration of the curcuminoid or the pharmaceutically active salt, metabolite, or analog thereof.

42. The method of claim 41, wherein the topical preparation contains a picomolar or nanomolar concentration of the curcuminoid or the pharmaceutically active salt or metabolite thereof.
